(19) United States
(12) Patent Application Publication
(10) Pub. No.: US 2013/0333460 A1
(43) Pub. Date: Dec. 19, 2013

(54) METHOD AND SYSTEM FOR MEASURING THE COMPOSITION OF A MULTIPHASE WELL SAMPLE

(71) Applicant: Frank Edward Bergren, GS Rijswijk (NL)

(72) Inventor: Frank Edward Bergren, GS Rijswijk (NL)

(21) Appl. No.: 13/996,460

(22) PCT Filed: Dec. 23, 2011
(86) PCT No.: PCT/EP2011/073936

§ 371 Date: Aug. 29, 2013

(30) Foreign Application Priority Data
Dec. 24, 2010 (EP) 10196996.2

(51) Int. Cl. G01N 33/24

(52) U.S. Cl. CPC G01N 33/246
USPC 73/152.42

(57) ABSTRACT

The accuracy of a measurement of the composition of a multiphase sample containing crude oil and water is improved by pouring the sample into a bottle shaped container (1), which is dimensioned such that the oil-water interface (4) is located in an upper portion (3), which forms a bottleneck that has a smaller cross section than a lower portion (2) of the container (1), which lower portion (2) has a substantially frusto-conical shaped intermediate section (8) contiguous with the upper portion (3) that allows accurate water cut measurement even if the oil-water interface is located within the intermediate section (8).

### BACKGROUND OF THE INVENTION

The invention relates to a method and system for measuring the composition of a multiphase well sample containing crude oil and water.

It is known to measure the composition of a multiphase well sample by pouring the sample into a substantially cylindrical transparent container, which is rotated by a centrifuge such that the oil and water phases are separated from one another, whereupon the oil and water content of the sample are estimated by measuring the height of the stacked oil and water columns in the container.

It is furthermore known from U.S. Pat. Nos. 3,712,118 and 4,388,407 to use bottle shaped transparent containers, which comprise trumpet-shaped transition sections between the large diameter bottom section and small diameter top section of the container, so that in case the oil interface is located in the trumpet-shaped transition section it is difficult to make an accurate assessment of the water cut of the multiphase well sample.

There is a need for a method and system that allows the measurement of the oil and water content of a multiphase well effluent sample more accurately in cases where the water is by far the predominant phase.

### SUMMARY OF THE INVENTION

In accordance with the invention there is provided a method for measuring the composition of a multiphase well effluent sample containing crude oil and water, the method comprising:
pouring the sample into a container with an at least partly transparent side wall that extends between the top and bottom of the container;
allowing the crude oil and water phases in the sample to separate such that a visible oil-water interface is formed within the container; and
determining the relative fraction of each phase in the sample by visually comparing the location of the oil-water and other interfaces against graduated volume markings on the at least partially transparent side wall of the container;
wherein the accuracy of the measurement is enhanced, in particular for samples in which the water is by far the predominant phase, by:
providing the container with a bottle-shaped profile such that a lower portion of the container, which is located adjacent to the bottom of the container, has a larger cross section than an upper portion of the container, which is located adjacent to the top of the container;
characterized in that the accuracy of the measurement is further enhanced by providing the lower portion of the container with a substantially frusto-conical shaped intermediate section contiguous with the upper portion. It will be understood that the presence of the frusto-conical shaped intermediate section allows a more accurate water cut measurement than the known trumpet-shaped intermediate sections known from U.S. Pat. Nos. 3,712,118 and 4,388,407 in case the oil-water interface is located within the intermediate section.

Optionally, the steps of allowing the oil and water phases to separate comprise:
heating the sample; and/or
placing the container in a centrifuge such that the bottom is located at a greater distance from an axis of rotation of the centrifuge than the top; and
rotating the centrifuge and container about the axis of rotation.

Also, optionally, minute amounts (generally <0.1% of the total volume) of chemical, often called demulsifier, can be added to assist phase separation.

The composition of the well sample determined by using the method according to the invention may be used to assess the relative amounts of crude oil and water flowing from a well that transects a natural subterranean reservoir, from which the sample is taken to manage and optimize the production of crude oil and other fluids from the reservoir. Similarly, the method can be used to assess the relative amounts of crude oil and water flowing in the combined fluids from several wells transecting a subterranean reservoir or reservoirs when the sample has been taken from an appropriate location in the gathering system.

In accordance with the invention there is furthermore provided a system for measuring the composition of a multiphase well sample containing crude oil and water, the system comprising a container having an at least partly transparent side wall extending between the top and bottom of the container, which container:
has a lower portion, which is located adjacent to the bottom of the container, having a larger cross section than an upper portion of the container, which is located adjacent to the top of the container;
characterized in that the lower portion of the container comprises a substantially frusto-conical shaped intermediate section contiguous with the upper portion.

The side wall of the container according to the invention may have a substantially tubular shape and the bottom portion of the container may have a larger internal diameter than the top portion of the container.

It is preferred that the top portion forms a bottleneck, of which the internal diameter is at least 10% smaller than the internal diameter of the lower portion and which extends along at least 10% of a distance between the top and bottom of the container.

The container or at least part of the at-least partly transparent side wall of the container may be made of transparent plastic or glass.

The system according to the invention may furthermore comprise a centrifuge with means for supporting the container such that when the centrifuge rotates about an axis of rotation the bottom of the container is located at a greater distance from the axis of rotation than the top of the container.

The means for supporting the container may comprise a pivot assembly which is clamped to the upper portion of the container and which permits a longitudinal axis, which extends between the top and the bottom of the container, to have a substantially vertical orientation when the centrifuge does not rotate and to have a substantially radial orientation relative to the axis of rotation when the centrifuge rotates.

These and other features, embodiments and advantages of the method and system according to the invention are described in the accompanying claims, abstract and the following detailed description of non-limiting embodiments depicted in the accompanying drawings, in which description reference numerals are used which refer to corresponding reference numerals that are depicted in the drawings.

Similar reference numerals in different figures denote the same or similar objects.

### BRIEF DESCRIPTION OF THE DRAWINGS

FIG. 1 shows a bottle shaped container according to the present invention; and

FIG. 2 shows a substantially cylindrical container according to the prior art

### DETAILED DESCRIPTION OF THE DEPICTED EMBODIMENTS

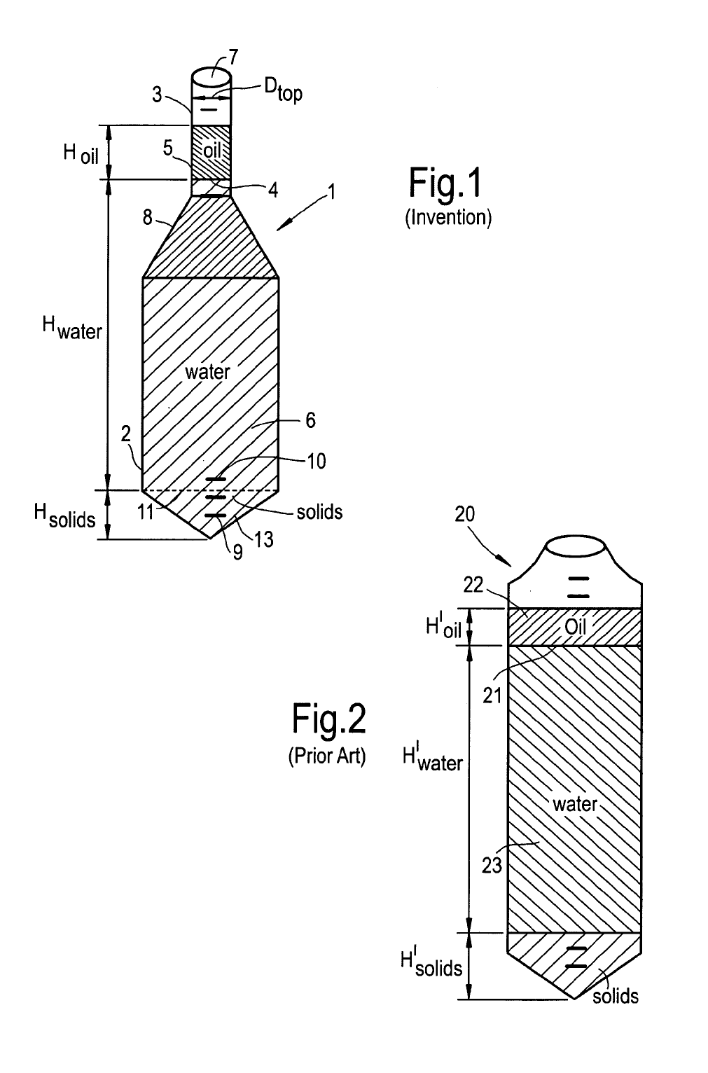

FIG. 1 shows a bottle shaped container 1 according to the invention, which comprises a large diameter lower section 2 and a small diameter upper section 3, connected by a tapering cylindrical bottleneck. The upper section has a diameter Dₜₒₚ, which is at least 10% smaller than the diameter D_{bottom} of the lower section.

The container shown in FIG. 1 furthermore has an open top 7, a frusto-conical mid section 8, a bottom 9, which is in the example shown conical, but may have any other suitable shape, and a series of graduation lines 10 which enable measurement of the relative volumes of the oil, water and solids in the sample from the location of the oil-water interface 4, the solids-water interface 11 and the oil surface 12 above the bottom of the container 9.

The dimensions of the upper and lower sections 2 and 3 of the container 1 according to the invention are selected such that the oil-water interface 4 between the oil phase 5 and the water phase 6 is located in the small diameter top section 2 for samples in which water is by far the predominant phase. This requires making an estimate of the relative volume fractions of oil and water in the sample from the multiphase well sample and then designing the container 1 such that the water and solid phases 6 and 13 entirely fill the interior of lower section 2 and of at least part of the upper section 3.

In the example shown in FIG. 1 the top section 3 has a diameter Dₜₒₚ which is less than 50% of the diameter D_{bottom} of the bottom section 3. It will be understood that since the oil fraction 5 is entirely contained in the top section 3 the volume of the oil column Hₒᵢₗ can be measured accurately, so that the relative volume fraction of oil in the total sample, often called “the oil cut” and the relative volume fraction of solids in the total sample can also be monitored accurately by visually noting the volumes of the oil, water and solid columns against the graduations on the container.

FIG. 2 shows a conventional substantially cylindrical container 20 according to the prior art. The known container is shaped such that the oil-water interface 21 between the oil and water fractions 22 and 23 is located in a substantially cylindrical part of the container 20 for all cases except when the relative volume fractions of both the solids and water are small.

The height of the oil, water and solid columns H′ₒᵢₗ, H′_{water} and H′_{solids} in the cylindrical container shown in FIG. 2 does not allow an enhanced accuracy of the measurement of the relative volume fraction of the water and oil content of the sample as provided by the container 1 according to the invention shown in FIG. 1.

The container 1 according to the invention as shown in FIG. 1 allows the measurement of the relative volume fraction of water and oil of the sample with enhanced accuracy, which is particularly important if the oil is produced through wells in which water is the predominant phase, typically in which water comprises more than 90 Vol % of the liquids. In such case it is important to measure the relative volume fraction of water and oil content with an inaccuracy of less than 1 Vol %. It will be understood that if a first crude oil well produces 95 Vol % water and a second crude oil well produces 97.5% water and both wells produce the same flux of well effluents, crude oil production from the second well is 50% of the crude oil production from the first well.

The dimensions of the container 1 according to the invention as shown in FIG. 1 can be configured by a person skilled in the art such that the accuracy of the measurement of the relative volume fractions of water and oil of the sample can be improved significantly using no more sophisticated technique than visually noting the interfaces 4 and 11 versus graduations 10 and the inaccuracy of the measurement is less than a few percent, preferably less than 1%.

1. A method for measuring the composition of a multiphase sample containing crude oil and water, the method comprising:
pouring the sample into a container with an at least partly transparent side wall that extends between the top and bottom of the container;
allowing the crude oil and water phases in the sample to separate such that a visible oil-water interface is formed within the container; and
determining the relative fraction of each phase in the sample by visually comparing the location of the oil-water and other interfaces against graduated volume markings on the at least partially transparent side wall of the container;
wherein the accuracy of the measurement is enhanced by:
providing the container with a bottle-shaped profile such that a lower portion of the container, which is located adjacent to the bottom of the container, has a larger cross section than an upper portion of the container, which is located adjacent to the top of the container;
characterized in that the accuracy of the measurement is further enhanced by providing the lower portion of the container with a substantially frusto-conical shaped intermediate section contiguous with the upper portion.

2. The method of claim 1, wherein the profile of the container has a substantially tubular shape and the lower portion of the container has a larger internal diameter than the upper portion of the container.

3. The method of claim 2, wherein the upper portion of the container forms an elongate bottleneck, of which the internal diameter is at least 10% smaller than the internal diameter of the lower portion and which extends along at least 10% of the distance between the top and bottom of the container.

4. The method of claim 2, wherein the dimensions of the upper portion and the lower portion are selected such that the oil-water interface is located in the upper portion or intermediate section of the container.

5. The method of claim 1, wherein the step of allowing the oil and water phases to separate comprises:
heating the sample; and/or
adding a demulsifier to the sample and/or;
placing the container in a centrifuge such that the bottom is located at a greater distance from an axis of rotation of the centrifuge than the top; and
rotating the centrifuge and container about the axis of rotation.

6. The method of claim 1, wherein the determined composition of the sample is used to assess the relative amounts of crude oil and water flowing from a well that transects a natural subterranean crude-oil containing reservoir from which the sample is taken and to manage and optimize the production of crude oil and other fluids from the reservoir.

7. A system for measuring the composition of a multiphase sample containing crude oil and water, the system comprising a container having an at least partly transparent side wall extending between the top and bottom of the container, which container has a lower portion, which is located adjacent to the bottom of the container, having a larger cross section than an upper portion of the container, which is located adjacent to the top of the container;
characterized in that the lower portion of the container further comprises a substantially frusto-conical shaped intermediate section contiguous with the upper portion.

8. The system of claim 7, wherein the profile has a substantially tubular shape and the lower portion of the container has a larger internal diameter than the upper portion of the container.

9. The system of claim 8, wherein the upper portion forms a bottleneck, of which the internal diameter is at least 10% smaller than the internal diameter of the lower portion and which extends along at least 10% of a distance between the top and bottom of the container.

10. The system of claim 7, wherein the container has an open top.

11. The system of claim 7, wherein at least the at least partly transparent side wall of the container is made of transparent plastic or glass.

12. The system of claim 7, wherein the container is made of transparent plastic or glass.

13. The system of claim 7, further comprising a centrifuge with means for supporting the container such when the centrifuge rotates about an axis of rotation the bottom of the container is located at a larger distance from the axis of rotation than the top of the container.

14. The system of claim 13, wherein the means for supporting the container comprises a pivot assembly which is clamped to the upper portion of the container and which permits a longitudinal axis, which extends between the top and the bottom of the container, to have a substantially vertical orientation when the centrifuge does not rotate and to have a substantially radial orientation relative to the axis of rotation when the centrifuge rotates.
